(19) United States
(12) Patent Application Publication
(10) Pub. No.: US 2017/0080026 A1
(43) Pub. Date: Mar. 23, 2017

(54) METHOD AND COMPOUND FOR OPENING POTASSIUM CHANNELS OF MAMMAL VASCULAR SMOOTH MUSCLES IN A REMOTE CONTROL MANNER

(71) Applicant: Tovaristvo Z Obmezhenou Vidpovidalnistu "Nanomedtrast", Kyiv (UA)

(72) Inventors: Anatoly I. Soloviev, Kyiv (UA); Oleksandr V. Zholos, Kyiv (UA); Irina V. Ivanova, Kyiv (UA); Tetiana V. Novokhatska, Kyiv (UA); Oleksandr L. Stroyuk, Kyiv (UA); Oleksandra Ye. Raievska, Kyiv (UA); Igor M. Parneta, Kyiv (UA); Volodymyr S. Yefanov, Kyiv (UA)

(21) Appl. No.: 15/259,610

(22) Filed: Sep. 8, 2016

(30) Foreign Application Priority Data
Sep. 10, 2015 (UA) A201508753

(51) Int. Cl. A61K 33/24; A61N 5/06; A61K 9/16

(52) U.S. Cl. CPC A61K 33/24; A61K 9/16; A61N 5/062; B82Y 5/00

(57) ABSTRACT

A claimed invention relates to the field of medicine or biology, namely the methods using nanotechnology objects, in particular colloidal solutions of gold nanoparticles which demonstrate a strongly marked effect on cellular proteins and can be used to restore the contractile function of mammalian vascular smooth muscle cells. Further, the invention relates to medicinal products which comprise gold nanoparticles and may be used for remote opening of voltage-gated ion channels in mammalian vascular smooth muscle cells.

A method for remote opening of voltage-gated ion channels in mammalian vascular smooth muscle cells comprises extracellular application of a colloidal solution of gold nanoparticles in population of mammalian vascular smooth muscle cells.

A medicinal product opening voltage-gated ion channels in mammalian vascular smooth muscle cells comprises the colloidal solution of gold nanoparticles at a concentration required for remote opening of voltage-gated ion channels in mammalian vascular smooth muscle cells.

### FIELD OF AN INVENTION

A claimed invention relates to the field of medicine or biology, namely the methods using nanotechnology objects, in particular colloidal solutions of gold nanoparticles which demonstrate a strongly marked effect on cellular proteins and can be used to restore the contractile function of mammalian vascular smooth muscle cells. Further, the invention relates to medicinal products which comprise gold nanoparticles and may be used for remote opening of voltage-gated ion channels in mammalian vascular smooth muscle cells.

### BACKGROUND OF THE INVENTION

Many types of nanomaterials are now extensively used in medicine and biology, such as biosensors, biomaterials, drug delivery systems and the like (Yang et al, 2010); however they are not used as medicinal products. It is apparent that properties of nanoparticles (NPs) may substantially vary from those of the same material when it is available in a macroform. These new physical and/or chemical properties are found in unexpected biological or even cytotoxic effects (Seaton et al, 2010). Because of high reactive ability of nanoparticles to interact with cellular proteins combined with their extremely small size and, therefore, better ability to penetrate cell structures, it becomes clear that understanding of how nanoparticles interact with living cells is important for theoretical and clinical medicine and biology.

The contractile function of smooth muscle (SM) cells is very important in all aspects of human physiology since these cells are the main element of effector hollow organs, including blood vessels. Therefore, SMs play a key role in supporting human life in a normal and pathological condition because they provide adequate supply of blood and oxygen.

The most important and widespread cardiovascular diseases, such as hypertension (Cox, Folade, Swanson, 2001; Novokhatska, Tishkin, Dosenko et al, 2013) ionizing radiation-induced (Soloviev, Tishkin, Kyrychenko, 2009) and diabetes-induced angiopathies (Klymenko, Novokhatska, Kizub et al, 2014), were supposed to be associated with excessive reactive oxygen intermediates (ROIs) and consequent protein kinase C activation. Basic mechanisms include disorder of vasodilating potential caused by reducing endothelium-dependent vasorelaxation (Soloviev et al, 2003; Ivanova et al, 2012) and/or inhibiting ion currents through potassium (K⁺) channels (Soloviev, Tishkin, Ivanova et al, 2009; Soloviev, Tishkin, Kyrychenko et al, 2009; Kizub et al, 2010; Kyrychenko et al, 2010). Finally, all these factors taken together contribute to vascular insufficiency and reduce blood supply to tissues. SM contractility is known to closely relate to their membrane potential, which in turn is determined preferably by K⁺ membrane conductivity. Therefore, K⁺ conductivity is generally thought to play an important role in regulation of SM cell membrane potential and, therefore, regulation of vascular tone. Four basic types of K⁺ channels in SM cells have been described: voltage-gated K⁺ channels (Kv) encoded by Kv gene family, inwardly(abnormally)-rectifying K⁺ channels (KIR) encoded by Kir2.0 gene, atropine-sensitive K⁺ channels (K_{ATP} encoded by Kir6.0 and genes encoding sulfonylurea receptor expression, and high-conductivity Ca²⁺-dependent K⁺ channels (BK_{Ca}) encoded by gene Slo1 (KNCMA1). At the molecular level, BK_{ca} channels comprise pore-forming alpha subunits and regulatory B1 subunits. The presence of B1 subunits provides higher Ca²⁺ sensitivity to BK_{ca} channel, making said channel an effective modulator of SM vascular function in healthy and sick subjects. The role of ion channels in SM contraction is usually assessed by pharmacological tools and patch-clamp technique recording membrane potential in various modifications. As was shown, BK_{ca} channels play an important role in the treatment of hypertension (Kyrychenko, Tishkin, Dosenko et al, 2012) and radiation-induced vascular hypertension (Kizub et al, 2010). The available data clearly indicates that reduction of outgoing current density in endothelial cells and SM cells combined with reduced BK_{Ca} mRNA expression led to vascular hypercontractility and the development of hypertension (Soloviev, Tishkin, Ivanova et al, 2009). It is important to note that, despite widespread use of nanoparticles including gold NPs (Au NPs), evidence of any direct effect of NPs on effector and regulatory elements of the vascular system, i.e. SM cells and endothelium, is not known in biology and medicine.

The object of this invention was to achieve the effect of relaxation in mammalian vascular smooth muscle cells using Au NPs and to establish mechanisms of changing outgoing ion currents and contractility in SM cells.

### DESCRIPTION OF THE PRIOR ART

The prior art discloses a method of SM cell relaxation in a subject, the method comprising administration of a complex containing choline conjugated Au NPs to a subject. The complex interacts with acetylcholine receptors of SM cells in a subject, and Au NPs in the complex bind to sulfhydryl receptor groups, thereby blocking the transmission of nerve impulses in subject's neuromuscular junction (U.S. Pat. No. 8,357,719 B2 published on Jan. 22, 2013).

The prior art also discloses a method of achieving the relaxing effect on muscles and treating atrium fibrillation using a complex containing negatively charged Au NPs having anionic bonds with choline or spermidine. Complexes migrate to an acetylcholine receptor with subsequent rupture of ionic bonds in the complex for stimulation of Au NP bond with Cys-loop acetylcholine receptors (Application U.S. 20120295969 A1 published on Nov. 22, 2012).

The prior art also discloses a method of remote control over neuronal activity using quantum dots (QDs) sensitive to optical excitation and nanoparticles of a semiconductor material. The method comprises activating voltage of closed ion channels using optically excited QDs. Fields formed by photogenerated QD dipoles excite cell membrane potential and create action potentials that control communication and signals between neurons. Such remote method for switching cell activity allows orienting specific cells by changing QD surface chemistry and interacting with specific proteins in cells (Lih Lin, Quantum dot optical switches for remote control of neurons, 30.04.2012, SPIE Newsroom).

Closer to the invention are the methods and compositions for remote control over cell functions which are based on the use of radio frequency waves from excitation of nanoparticles, in particular magnetite NPs targeted to specific types of cells. Magnetite NPs can be applied to a target cell ex vivo and/or for intracellular expression. Types of target cells have a sensitive temperature channel wherein excitation of nanoparticles leads to localised temperature rise causing cellular response. Such cellular responses may include, for example increased gene expression resulting from production by one or more physiologically active proteins. Expression of such proteins may be used to treat a variety of inherited or acquired diseases or disorders in a subject, in particular any disease associated with protein deficiency (publication of the international application WO 2013 029025 A1 dd. Feb. 28, 2013).

### SUMMARY OF THE INVENTION

The object of the invention is to provide remote activation of voltage-gated ion channels in mammalian vascular smooth muscle cells, in particular potassium channels, using gold nanoparticles. A further object is to enhance activation of voltage-gated ion channels in mammalian vascular smooth muscle cells which is obtained using gold nanoparticles through laser irradiation.

This object is achieved so that according to the invention the claimed method for remote opening of voltage-gated ion channels in mammalian vascular smooth muscle cells comprises extracellular application of the colloidal solution of gold nanoparticles in population of mammalian vascular smooth muscle cells.

According to one embodiment of the invention, the method may use high-conductivity potassium channels as voltage-gated ion channels.

According to an additional embodiment of the invention, the method may use the colloidal solution of gold nanoparticles 4 . . . 7 nm in size.

According to yet another additional embodiment of the invention, the method may use the colloidal solution of gold nanoparticles 5 nm in size.

According to yet another additional embodiment of the invention, the method may use the colloidal solution of gold nanoparticles having electrokinetic potential of at least 35 mV.

According to yet another additional embodiment of the invention, the method may use the colloidal solution of gold nanoparticles with maximum plasmon resonance absorption band within 510 . . . 570 nm.

According to yet another additional embodiment of the invention, the method may use the colloidal solution of gold nanoparticles at a concentration within 10⁻⁶ . . . 3×10⁻⁴ M.

According to yet another additional embodiment of the invention, the method may use the colloidal solution of gold nanoparticles stabilised with sodium ascorbate.

According to yet another additional embodiment of the invention, the method may further use laser irradiation of population of mammalian vascular smooth muscle vascular cells after extracellular application of the colloidal solution of gold nanoparticles.

According to yet another additional embodiment of the invention, the method may use laser irradiation having power of 5 mW and wavelength of 532 nm.

According to yet another additional embodiment of the invention, the method may use laser irradiation for at least 5 seconds.

Further, the said object may be achieved so that, according to the invention, the claimed medicinal product opening voltage-gated ion channels in mammalian vascular smooth muscle cells comprises the colloidal solution of gold nanoparticles at a concentration required for remote opening of voltage-gated ion channels in mammalian vascular smooth muscle cells.

According to one embodiment of the invention, the colloidal solution of gold nanoparticles may contain nanoparticles 4 . . . 7 nm in size.

According to the additional embodiment of the invention, the colloidal solution of gold nanoparticles may contain nanoparticles 5 nm in size.

According to the additional embodiment of the invention, the colloidal solution of gold nanoparticles may contain nanoparticles having electrokinetic potential of at least 35 mV.

According to the additional embodiment of the invention, the colloidal solution of gold nanoparticles may contain nanoparticles having maximum plasmon resonance absorption band within 510 . . . 570 nm.

According to the additional embodiment of the invention, the colloidal solution of gold nanoparticles may contain nanoparticles at a concentration within 10⁻⁶ . . . 3×10⁻⁴ M.

According to the additional embodiment of the invention, the medicinal product opening voltage-gated ion channels in mammalian vascular smooth muscle cells contains the colloidal solution of gold nanoparticles stabilised with sodium ascorbate.

### BRIEF DESCRIPTION OF THE DRAWINGS

The claimed invention is illustrated by the following exemplary embodiment of the method and a means of remote opening of voltage-gated ion channels in mammalian vascular smooth muscle cells as well as by drawings and images where:

FIG. 1 shows distribution of sodium ascorbate-stabilised colloidal solution of gold nanoparticles by hydrodynamic size. Insert: SEM microphotography of gold nanoparticles.

FIG. 2 shows integral curves concentration-relaxation following the action of Au NPs≈5 nm in size and exposure to “green” laser irradiation 5 mW at a wavelength of 532 nm in intact endothelium denuded rat thoracic aorta rings previously activated with 10⁻⁶ M norepinephrine before and after paxillin exposure (500 nM).

FIG. 3 shows relative (A) and original (B) curves of I-V outgoing currents recorded in isolated rat thoracic aorta smooth muscle cells used as control and after exposure to Au NPs (10⁻⁴ M) and following the administration of Au NPs in combination with exposure to “green” laser irradiation 5 mW at a wavelength of 532 nm. C, D show effects of potassium channel blockers (paxillin, 500 nM, and tetraethanolammonium 10 mm) on the outgoing potassium current in smooth muscle cells of thoracic aorta not exposed to Au NPs (C) and exposed to Au NPs (D). Currents caused by single step excitations with an amplitude of 10 mV and 300 ms increments within −100 and +70 mV of outgoing potential at −60 mV. These data represent mean values ±SEM (n=8).

Designation*−P <0.05 compared to the combined influence of Au NPs and laser irradiation.

FIG. 4 shows stable activation curves for Iₓ in isolated vascular smooth muscle cells used as control (squares), following by administration of Au NPs (10⁻⁴ M, circles) and following the combined action of Au NPs in combination with exposure to “green” laser irradiation 5 mW at a wavelength of 532 nm (triangles) defined with 300 ms increments. Currents in each cell (except for leakage current) were converted into specific electrical conductivity (pS/pF), averaged and depicted versus membrane potential (mV). The data thus obtained were brought in consistency using the standard Boltzmann function with average parameters described herein. The resulting current-voltage characteristics (curves I-V) were obtained by measuring current at the end of an impulse. The data represent mean values of ±SEM (n=8).

Designation*−P<0.05 compared to control, #−P<0.05 compared to Au NPs.

FIG. 5 shows enhanced Au NP effect which is visible by the activity of single BK_{Ca} channels in vascular smooth muscle cells in rats.
Panel A on FIG. 5 shows BK_{Ca} channel activity recorded at −20 mV (the upper panel) before and after (shown by a horizontal line at the upper panel) administration of Au NPs (10⁻⁵ M). Two ten-second segments of current lines defined at time points a and b along the entire time line (the upper panel) are shown on the extended time scale in the middle of the panel. In a study membrane fragment, up to 11 active channels were found in the full activation state as demonstrated by numerous holes shown by horizontal dotted lines. The lower figure shows NPo value defined during successive five-second intervals. Panel B on FIG. 5 shows BKCa channel activity recorded at 40 mV in another small fragment of plasma membrane whereon electrode was placed, and Au NPs were first applied at a concentration of 10⁻⁵ M followed by 10⁻⁴ M (shown by thin horizontal lines). The result of additional exposure to “green” laser irradiation is showed by thick horizontal lines. The data was analysed and illustrated similar to panel A.

### DETAILED DESCRIPTION OF THE INVENTION

The effect of remote opening of voltage-gated ion channels in mammalian vascular smooth muscle cells by extracellular application of the colloidal solution of gold nanoparticles in the population of mammalian vascular smooth muscle cells was studied in 6-8 week old male rats Wistar (body weight: 250-350 g) which are held under controlled environmental conditions (+21° C., day-night cycle: 12 h/12 h) and have free access to water and standard food for rats.

For the purposes of the study, smooth muscle cells isolated from rat thoracic aorta using collagenase and pronase were taken. Rats were anesthetised with ketamine (37.5 mg/kg body weight, intraperitoneally) and xylazine (10 mg/kg body weight, intraperitoneally), and thoracic aorta about 1.0 . . . 1.5 cm long was removed and denuded to make it free from connective tissue. Aorta was cut into small pieces (1.5×1.5 mm) in the cooled low Ca²⁺ ion content solution comprising (in mM): NaCl 140, KCl 6, MgCl₂ 3, D-glucose 10, HEPES 10 (pH 7.4) for 15 minutes. Vascular tissues were placed in fresh low Ca²⁺ ion content solution comprising collagenase type IA 2 mg/mL (417 U/mg), pronase type E XXV 0.5 mg/ml, and bovine albumin serum 2 mg/ml. Then tissues were incubated for 30 minutes at a temperature of +37 ° C. After incubation, tissues were washed (for 2-3 minutes) twice in fresh low Ca²⁺ ion content solution to remove enzymes. Cells were dispersed by stirring using a glass pipette and then placed in modified Krebs bicarbonate buffer. A certain number of muscle cells was maintained at a temperature of +40° C. and remained functional for at least 4 hours.

### Results

The studies were conducted using Au NP colloidal solutions with different NP size and various stabilisers; however ascorbate-stabilised Au NPs 4 . . . 7 nm in size, more preferably ≈5 nm in size, demonstrated strongly marked ability to relax vascular smooth muscle cells in rats by remote control (see Table 1 below).

TABLE 1
Vasodilating action of Au NP colloidal solution with NPs of different size and stabilizers

| | | | Maximum | Maximum | |
|---|---|---|---|---|---|
| | | | relaxing | relaxing | |
| | | | effect | effect | |
| | | | achieved by | achieved by | |
| | Au NP | Composition of the | Au NPs | stabiliser | |
| # | size | stabiliser | (10⁻⁴M), % | (10⁻⁴M), % | Conclusion |
| 1 | 7 nm | Trisodium citrate 1 g/l | 44.1 | 40.3 | Vasodilating |
| | | Sodium oxalate 420 mg/l | | | effect is |
| | | | | | achieved mainly |
| | | | | | by action of the |
| | | | | | stabiliser. |
| 2 | 15 nm | Sodium oxalate 80 mg/l | 2.1 | — | No vasodilating |
| | | Sodium acetate 300 mg/l | | | effect is |
| | | Sodium chloride 200 mg/l | | | observed. |
| | | Ethanol 1% | | | |
| 3 | 4 nm | Sodium acetate 300 mg/l | 51.2 | 43.7 | Vasodilating |
| | | Sodium chloride 200 mg/l | | | effect achieved |
| | | Sodium oxalate 420 mg/l | | | by the stabiliser |
| | | Sodium borate 5 mg/l | | | was too strong. |
| 4 | 5 nm | Sodium acetate 300 mg/l | 55.3 | — | Best relaxation |
| | | Sodium chloride 200 mg/l | | | ability |
| | | Sodium oxalate 420 mg/l | | | |
| | | (ascorbate | | | |
| | | oxidation product) | | | |

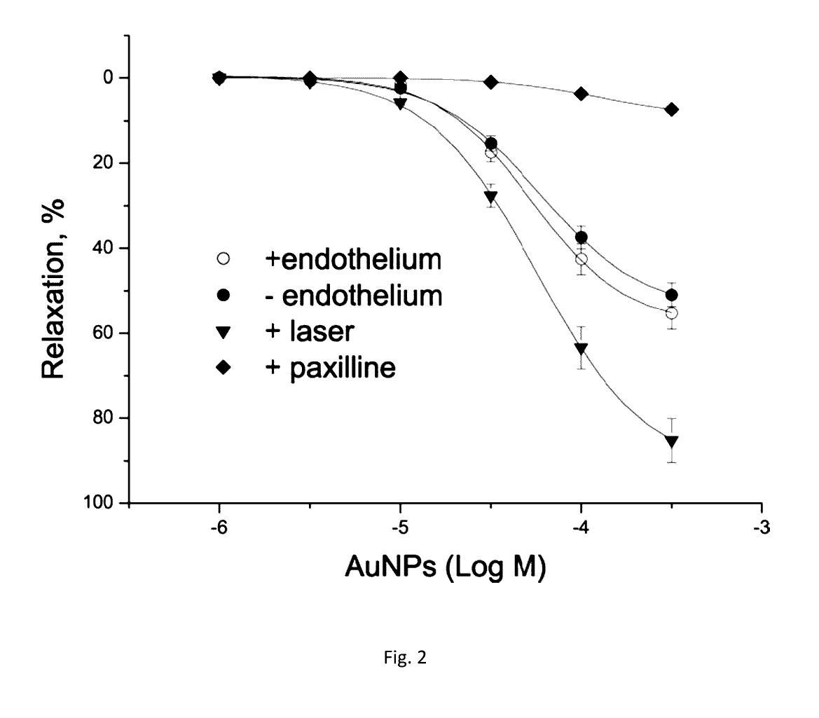

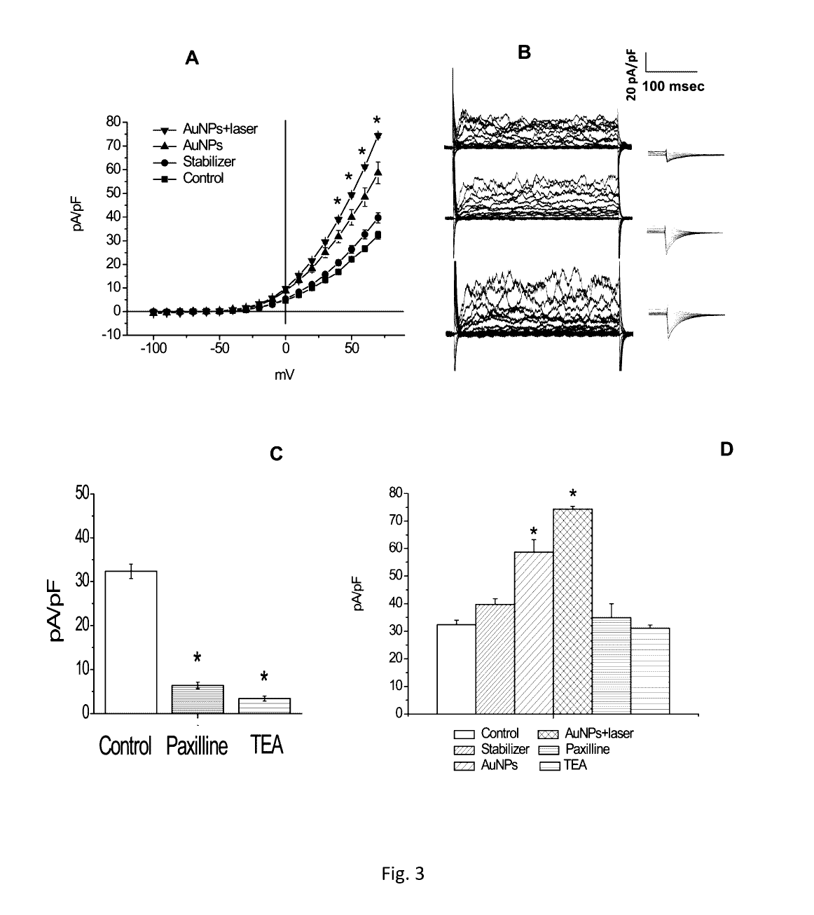

FIG. 2 shows dose-effect curves for Au NPs 5 nm in diameter in rat thoracic aorta SM narrowed by norepinephrine (NE, 10-6 M). Au NPs at a concentration range between 10⁻⁶ and 3×10⁻⁴ M apparently have the ability to significantly reduce the amplitude of norepinephrine-induced contraction in a dose-dependent manner (pD₂=4.2±03, Eₘₐₓ=55±4%), i.e. Au NPs demonstrated strongly marked vasodilating activity. Endothelial disorder had no significant impact on Au NPs induced SM relaxation. It is important to note that stabilisers alone had no effect on the tone of thoracic aorta SM narrowed with norepinephrine. To induce excitation of surface plasmon resonance in gold NPs, a cell with organ tissues was irradiated with “green” laser 5 mW at a wavelength of 532 nm over the entire period of Au NPs induced relaxation. In this case, addition of Au NPs resulted in a significant increase in the amplitude of maximum Au NP induced relaxation, from 55%±4 to 85±5.0% (n=10, p<0.05), while SM sensitivity to Au NPs did not increase, and the average value of pD₂ was ±0.013 (n=10, P>0.05). It is important to note that intensity of laser irradiation was so that it did not lead to any significant changes in temperature of Krebs solution in a bath. In the presence of paxillin (500 nm), a powerful BK_{Ca} channel blocker, Au NPs lost their vasodilating activity in isolated aortic SM segments almost completely (FIG. 2) demonstrating the critical involvement of BK_{Ca} channels in this case. The next panel of tests was conducted to study original K⁺ channel activity in control thoracic aorta SM cells. Single cells isolated enzymatically from healthy rat thoracic aorta were kept at resting potential −60 mV, and outgoing currents were then caused by stepwise increase of depolarizing voltage. The respective original current curves and current-voltage (I-V) relationship are shown on FIG. 3. The total amplitude of outgoing current (I_{K}) density in an intact SM cell was on average 32±2 pA/pF (n=12) at +70 mV. All cells clearly demonstrated marked trace currents confirming that measured currents were recorded mainly from external potassium channels (I_{K}). Further, spontaneous current fluctuations on a horizontal site of curve I_{K} resembled spontaneous transient outgoing currents (STOCs) placed on the total current of BK_{Ca} induced activation (FIG. 3B). Current to voltage ratio (curve IV) shows that outgoing currents in intact aorta cells are absent at potentials below −40 mV and markedly rise in response to exposure of depolarizing voltage. Curve IV shows nonlinear dependence at potentials above −40 mV which can be more clearly traced at more positive potentials than +25 mV (FIG. 3A). It is important to note that outgoing currents are activated quite slowly, and in control conditions inactivation was poor or was not observed during 300 ms impulses.

FIG. 3 A, B shows that the original curve IK and the curve IV obtained for SM cells treated with Au NPs (10⁻⁴ M) showed a significant increase in amplitude K⁺ current density from 32±2 pA/pF to 59±5 pA/pF+70 mV, respectively (P<0,05, n=10). External irradiation with “green” laser increased Au NP-induced increment I_{K} from 59±pA/pF to 74±1 pA/pF (n=10, P<0.05).

After that, various components of total potassium current activated by action of Au NPs were determined pharmacologically (FIG. 3C, D). It is clear that total outgoing current in rat thoracic aorta SM cells, mainly due to current activation, is provided through paxillin-sensitive BK_{Ca} channels. This conclusion is based on the following evidence. Paxillin (500 nM) added to external solution in a bath reduced outgoing current in untreated rat thoracic aorta SM cells significantly (from 32±2 to ±6 1 pA/pF, n=10, p<0.05). Additional administration of tetraethylammonium (10 mm) resulted in a decrease of outgoing current to 3±1 pA/pF (n=10, P<0.05). Application of paxillin (as well as TEA) stopped Au NPs-induced I_{K} increment (FIG. 3 D).

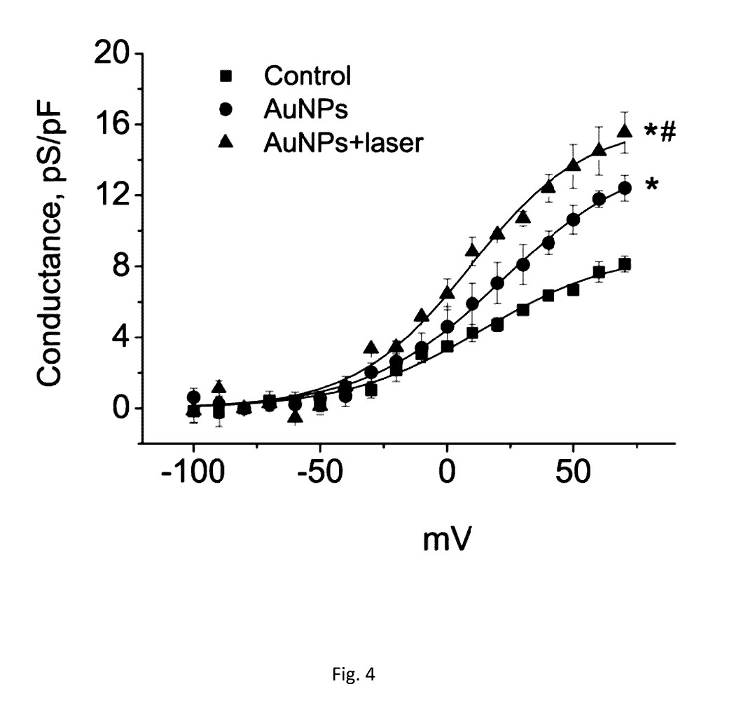

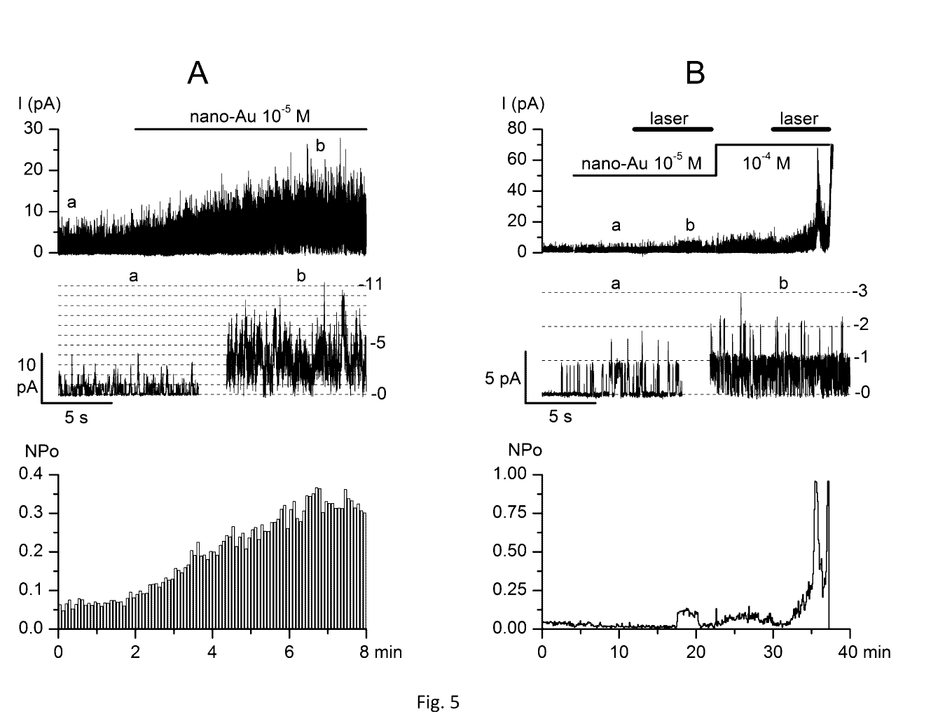

It is known that the relationship between potential applied to the channel and its current (conductivity) is described by Boltzmann equation. Thus, the standard Boltzmann equation can be used for static assessment of the impact of Au NPs on aortic Iₖ of SM cells before and after administration of Au NPs (FIG. 4). The curve conductivity-voltage thus built shows the nature of channel opening, specifically movement of charges in an area of channel protein sensitive to potential changes. Thus, values of fixed data points of activation correspond to Boltzmann equation, allowing to obtain half-maximal activation voltage (V_{1/2}) and showing a significant increase in values V₁₁₂ for Au NPs and combinations of Au NPs and “green” laser irradiation (V₁₁₂=8.5±0.5 and 14±0.8 MV, control condition and upon Au NP administration, respectively, n=10, p<0.05). Laser irradiation enhanced this effect (V₁₁₂=16+0.8 mV, n=10, P<0.05). These results clearly show that K⁺ channel activation after Au NP administration does not switch the general BK_{Ca} activation mechanism through a negative shift in the activation curve (i.e. changing voltage sensitivity was achieved by growth of free intracellular calcium concentration), and increasing conductivity of the entire cell is likely associated with changes in the channel conductivity (e.g. increasing the amplitude of single current or probability of channel opening (Pₒ)). The data shows a significant increase in conductivity by activating K⁺ channel in the whole range of potentials above −50 mV.

This effect was studied experimentally using single channel recordings, the most direct approach to establish a mechanism of channel modulation by Au NPs. In supracellular areas formed on rat aortic myocytes, activity of 3-5 BK_{Ca} channels typically occurs at potentials ranging from −20 to +40 mV. Depending on the extent of channel activity in each separate membrane segment, the optimal confining potential was set within this range so that the initial channel activity (typically expressed in NPo) would not be too high, for example about 0.05-0.1. Adding Au NPs potentiates BK_{Ca} activity with a delay for 1-2 minutes (FIG. 5 A), as it was originally observed for more frequent openings of channels with subsequent progressive emergence of additional open levels corresponding to multiple openings of channels with identical single-channel current amplitudes (FIG. 5 A, the middle panel). In the presence of Au NPs for 10-15 minutes and especially in combination with “green” laser irradiation, a significant increase in channel activity is observed with activation of more than 10 channels (e.g., FIG. 5 V, the upper curve, recording for about 35 minutes). Accurate measurement of NPo in such high channel activity is problematic due to emergence of noises on the curve; however 2-11-fold increase of NPo values was observed in all 3 sites obtained depending on the Au NP concentration irrespective of whether laser irradiation was applied or not. Therefore, Au NP impact on channel activity likely depends on their concentration (FIG. 5B); however it is difficult to quantify the maximum effect, especially at a concentration of 10⁻⁴ M, more precisely due to the above-mentioned problems of extremely high channel activity and a large number of channels in membrane areas. In terms of evaluation, mean fold increase in channel NPo was on average 3.4±0.7 at an Au NP concentration of 10⁻5 M and 8.5±2.5 at a concentration of 10⁻⁴ M, which indicates a dependence of Au NP opening effect on Au NP concentration. Similar results were obtained for BK_{ca} channels in mouse iliac myocytes (n=3, data not shown), indicating the effect of Au NPs as “openers” of BKCa channels both in vascular and visceral smooth muscle.

The mechanism of interaction Au NPs with potassium channels and strengthening of the achieved effect by exposing cells to laser irradiation may be associated with plasmon resonance effect, i.e. excitation of surface plasmons on the surface of Au NPs, which may be increased by irradiation with light at a resonance frequency. Au NPs, as nanosized structures of a noble metal, are known to have a distinctive feature, such as collective oscillation of electronic “gas” on NP surface (surface plasmon resonance) leading to photo-induced local electric fields near the surface of nanoparticles. In case of laser irradiation, this local electric field can be significantly enhanced by local plasmon resonance, which in turn affects ion channel voltage sensor, increases outgoing current, and leads to SM relaxation. In the absence of external irradiation, such plasmon may be excited by chemiluminescence of natural tissue.

The strength of an electric field in a cell membrane depends on Au NP concentration, and K⁺ channels may be activated when threshold Au NP concentration is reached.

Therefore, channel recordings shown on FIG. 5 provide direct confirmation of single-channel BK_{Ca} activation using Au NPs. “Green” laser irradiation is an additional intensifying factor in the presence of Au NPs and is ineffective in the absence of gold NPs.

### EXEMPLARY EMBODIMENT OF THE INVENTION

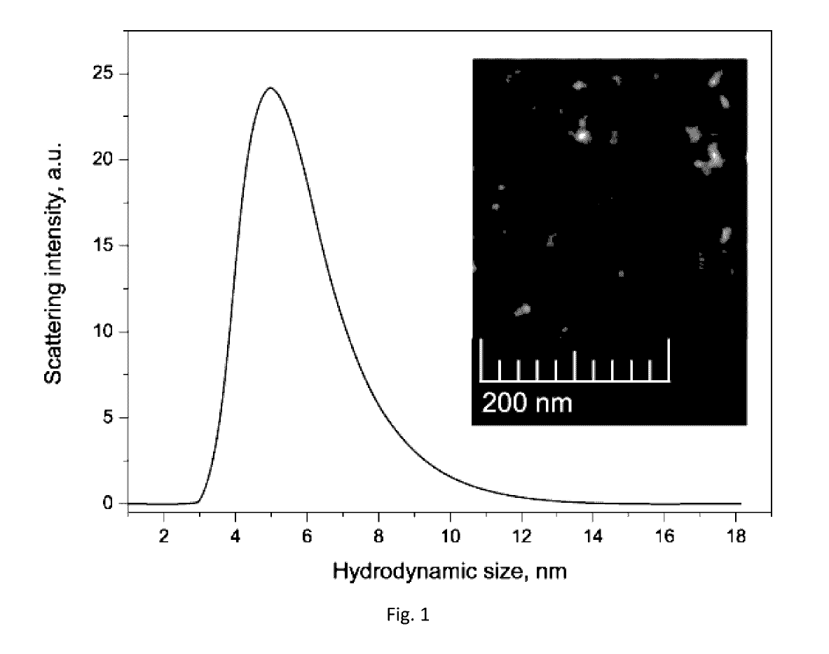

The medicinal product opening voltage-gated ion channels in mammalian vascular smooth muscle cells containing the colloidal solution of gold nanoparticles may prepared as follows. The Au NP colloid solution with maximum plasmon resonance absorption band within 532 nm is synthesised by reducing sodium tetrachloroaurate with sodium ascorbate in aqueous solutions at a room temperature. Colloidal solutions thus prepared are then neutralised with acetic acid to pH 6-7. Dynamic light scattering spectroscopy of the Au NP colloidal solution thus obtained (200 mg/l, i.e. ˜10⁻³ M or 6.02×10²⁰ particles/l) shows that the average hydrodynamic size of Au NPs is approximately 5 nm (FIG. 1). This result is confirmed by SEM method (see a fragment of SEM microphotography on the insert to FIG. 1). Au NP surface stabilised with ascorbate anions are negatively charged and provide electrokinetic (zeta) potential −35 mV to NP surface. The colloidal solutions thus prepared remained stable for many months and did not show noticeable changes in Au NP size and distribution by size over time. Following extracellular application of the colloidal solution of gold nanoparticles, population of mammalian vascular smooth muscle cells may be exposed to pulsed nanosecond irradiation allowing to increase irradiation capacity without side effects. According to another embodiment, fibre optic devices for endoscopic or interstitial irradiation may be used for irradiation (Dykman, Klebtsov, 2012). The claimed invention helps to achieve the effect of relaxation of mammalian vascular smooth muscle cells when Au NPs are used as colloidal solution with NPs 4 . . . 7 nm in size by remote opening of voltage-gated ion channels (in particular BK_{Ca} channels). The claimed method helps to renew normal vascular contractile activity in a number of diseases associated with the development of vascular hypercontractility in vascular SM.

### References:

1. Cox R, Folande K., Swanson R. Differential expression of voltage-gated K+ channel genes in arteries from spontaneously hypertensive and Wistar-Kyoto rats. Hypertension 2001; 3: 1315-1322.

2. Dykman L, Klebtsov N. Gold nanoparticles in biomedical applications: recent advances and perspectives. Chem Soc Rev. 2012; 41: 2256-2282.

3. Denson D D, Wang X, Worrell R T, Eaton D C. Effects of fatty acids on BK channels in GH3 cells. Am J Physiol. 2000; 279: C1211-C1219.

4. Eustis S, EI-Sayed M A. Why gold nanoparticles are more precious than pretty gold: Noble metal surface plasmon resonance and its enhancement of the radiative and nonradiative properties of nanocrystal of different shapes. Chem. Soc. Rev. 2006 ;35: 209-217.

5. Kizub I, Pavlova O, Ivanova I, Soloviev A. Protein kinase C-dependent inhibition of BKCa current in rat aorta smooth muscle cells following gamma-irradiation. Int J Radiat Biol 2010; 86: 291-299.

6. Kyrychenko S, Tishkin, S, Dosenko V, Ivanova I, Novokhatska T, Soloviev A. The BKCa channels deficiency as a possible reason for radiation-induced vascular hypercontractility. Vasc Pharmacol 2012; 56: 142-149.

7. Klymenko K, Novokhatska T, Kizub I, Parshikov A, Dosenko V, Soloviev A. PKC-6 isozyme gene silencing restores vascular function in diabetic rat. Bas Clin Physiol Pharmacol 2014; 27:1-9.

8. Lugo K, Miao, X, Rieke F, Lin L. Y. Remote switching of cellular activity and cell signaling using light in conjunction with quantum dots. Biomed Optics Express. 2012; 3: 447-454.

9. McGahon M K, Dash D P, Arora A, Wall N, Dawicki J, Simpson D A. Scholfield C N, McGeown J G, Curtis T M. Diabetes downregulates large conductance Ca²⁺-activated potassium beta 1 channel subunit in retinal arteriolar smooth muscle. Circ Res. 2007; 100(5):703-11.

10. Novokhatska T, Tishkin S, Dosenko V, Boldyrev A, Ivanova I, Strelkov I, Soloviev A. Correction of vascular hypercontractility in spontaneously hypertensive rats using shRNAs-induced delta protein kinase C gene silencing. Eur J Pharmacol 2013; 718: 401-407.

11. Seaton A, Tran L. Aitken R, Donaldson K.. Nanoparticles, human health hazard and regulation J R Soc Interface. 2010; 7: S119-S129. (doi:10.1098/rsif.2009.0252.focus).

12. Soloviev A, Tishkin S, Ivanova I, Zelensky S, Dosenko V, Kyrychenko S, Moreland RS. Functional and molecular consequences of ionizing irradiation on large conductance Ca²⁺-activated K⁺ channels in rat aortic smooth muscle cells. Life Sciences. 2009;84: 164-171.

13. Soloviev A, Tishkin S, Kyrychenko S., Quercetin-filled phosphatidylcholine liposomes restore abnormalities in rat thoracic aorta BKCa channel function following ionizing irradiation. Acta Physiol Sinica. 2009; 61 (3):201-210.

14. Soloviev A I, Tishkin S M, Parshikov A V, Ivanova I V, Goncharov E V, Gurney A M. Mechanisms of endothelial dysfunction after ionized irradiation: selective impairment of the nitric oxide component of endothelium-dependent vasodilatation. Br J Pharmacol 2003; 138: 837-844.

15. Yang Z, Liu Z W, Allaker R P, Reip P, Oxford J, Ahmad Z, Ren, G.. A review of nanoparticle functionality and toxicity on the central nervous system. J R Soc Interface. 2010; 2:1-12. (doi: 10.1098/rsif2010.0158.focus).

1. A method for remote opening of voltage-gated ion channels in mammalian vascular smooth muscle cells comprising extracellular application of a colloidal solution of gold nanoparticles in population of mammalian vascular smooth muscle cells.

2. The method according to claim 1 wherein high-conductivity potassium channels are used as voltage-gated ion channels.

3. The method according to claim 1 wherein said method uses the colloidal solution of gold nanoparticles 4 . . . 7 nm in size.

4. The method according to claim 1 wherein said method uses the colloidal solution of gold nanoparticles 5 nm in size.

5. The method according to claim 1 wherein said method uses the colloidal solution of gold nanoparticles having electrokinetic potential of at least 35 mV.

6. The method according to claim 1 wherein said method uses the colloidal solution of gold nanoparticles with maximum plasmon resonance absorption band within 510 . . . 570 nm.

7. The method according to claim 1 wherein said method uses the colloidal solution of gold nanoparticles at a concentration within 10⁻⁶ . . . 3×10⁻⁴ M.

8. The method according to claim 1 wherein said method uses the colloidal solution of gold nanoparticles stabilised with sodium ascorbate.

9. The method according to claim 1 wherein said method further comprises laser irradiation of the population of mammalian vascular smooth muscle vascular cells after extracellular application of the colloidal solution of gold nanoparticles.

10. The method according to claim 9 wherein said method uses laser irradiation having power of 5 mW and wavelength of 532 nm.

11. The method according to claim 9 wherein said method uses laser irradiation for at least 5 seconds.

12. A medicinal product opening voltage-gated ion channels in mammalian vascular smooth muscle cells comprising the colloidal solution of gold nanoparticles at a concentration required for remote opening of voltage-gated ion channels in mammalian vascular smooth muscle cells.

13. The medicinal product according to claim 12 wherein the colloidal solution of gold nanoparticles contains nanoparticles 4 . . . 7 nm in size.

14. The medicinal product according to claim 12 wherein the colloidal solution of gold nanoparticles contains nanoparticles 5 nm in size.

15. The medicinal product according to claim 12 wherein the colloidal solution of gold nanoparticles contains nanoparticles having electrokinetic potential of at least 35 mV.

16. The medicinal product according to claim 12 wherein the colloidal solution of gold nanoparticles contains nanoparticles with maximum plasmon resonance absorption band within 510 . . . 570 nm.

17. The medicinal product according to claim 12 wherein the colloidal solution of gold nanoparticles contains nanoparticles at a concentration of gold nanoparticles within 10⁻⁶ . . . 3×10⁻⁴ M.

18. The medicinal product according to claim 12 wherein said medicinal product contains the colloidal solution of gold nanoparticles stabilised with sodium ascorbate.
